(19) United States
(12) Patent Application Publication
(10) Pub. No.: US 2017/0138834 A1
(43) Pub. Date: May 18, 2017

(54) DEVICE AND METHOD FOR GAS AND PARTICLE MEASUREMENT

(71) Applicant: Robert Bosch GmbH, Stuttgart (DE)

(72) Inventor: Andreas Krauss, Tuebingen (DE)

(21) Appl. No.: 15/337,084

(22) Filed: Oct. 28, 2016

(30) Foreign Application Priority Data
Nov. 12, 2015 (DE) 102015222312.4

(51) Int. Cl. G01N 15/10; G01N 33/00

(52) U.S. Cl. CPC G01N 15/1056; G01N 33/0027

(57) ABSTRACT

A device and a method for gas or particle measurement in a portable electronic device. The portable device includes a housing which, with the exception of an overflow opening, has an almost gas- and air-tight design. A display unit, an operating unit, a transceiver unit, and optionally a power supply unit may be integrated into the housing in particular. Furthermore, the housing may also include the required electronics. By deflecting a movable outer wall of the housing with the aid of an actuator, the volume of the inner space of the housing may be increased or reduced. A gas exchange between the inner space and the space outside of the housing may thereupon take place via the overflow opening in the housing, and a possible substance, in particular, a predetermined gaseous component or a particle concentration may be analyzed in a gas flow during this gas exchange.

### CROSS REFERENCE

The present application claims the benefit under 35 U.S.C. §119 of German Patent Application No. DE 102015222312.4 filed on Nov. 12, 2015, which is expressly incorporated herein by reference in its entirety.

### FIELD

The present invention relates to a device and a method for gas or particle measurement. In particular, the present invention relates to the integration of a gas or particle measurement into a portable device of entertainment electronics.

### BACKGROUND INFORMATION

Surroundings sensors have lately been increasingly integrated into numerous electronic products such as, for example, cell phones, smart phones, etc. These products generally already contain a display unit, for example, an OLED or TFT display, an operating unit, for example, a keyboard or a touch screen, a transceiver unit, for example, for GSM, UMTS, LTE, Bluetooth, W-LAN, ZigBee. The individual components may be controlled with the aid of appropriate electronics. The electronics are formed, in particular, by a microprocessor-based structure. In this way, the integrated surroundings sensors and other components may be addressed, read out, and evaluated. Such surroundings sensors include, among others, dependent on a gas exchange with the surroundings. Such sensors may be, for example, pressure sensors, moisture sensors, gas detection sensors or sensors for measuring a particle concentration. For the function of sensors of this type it is necessary that a gas, such as the ambient air, for example, flow past the sensor relatively slowly.

The housings of electronic devices of this type, such as cell phones or smart phones, generally include a rigid front side having a glass-based display. The back side is usually formed by a flexible, or at least deformable, plastic or metallic cover. Housings of this type are generally very flat, i.e., the height of the housing is smaller by at least one order of magnitude than the width or length of the housing.

German Patent Application DE 10 2006 061 696 A1 describes a cell phone including an integrated gas sensor. The gas sensor is formed by a radiation source and a gas detector situated opposite the radiation source.

### SUMMARY

The present invention provides, according to one aspect, a gas or particle measuring device, and, according to another aspect, a method for gas or particle measurement.

A gas or particle measuring device including a housing, an actuator, a control device and a sensor is provided. The housing includes a movable outer wall. The movable outer wall is movable in relation to the remaining outer walls of the housing. Furthermore, the housing includes an overflow opening. The overflow opening is designed to enable a gas exchange between the inner space and the space outside of the housing. The actuator is designed to deflect the movable outer wall of the housing.

The control device is designed to control the actuator. The sensor is designed to detect a substance in a gas flow flowing through the overflow opening.

Furthermore, the following is provided:

A method for gas or particle measurement including the steps of providing a housing having an outer wall that is movable in relation to the rest of the housing, and of providing an actuator designed to deflect the movable outer wall. The housing provided also includes an overflow opening, which is designed to enable a gas exchange between the inner space and the space outside of the housing. The method further includes the steps of deflecting the movable outer wall of the housing with the aid of the actuator, and detecting a substance in a gas flow flowing through the overflow opening.

The present invention includes generating a pumping motion for a gas flow via targeted deflection of at least one outer wall of a housing. This pumping motion may be used for generating a targeted gas or air flow to a sensor, in particular, to a gas or particle sensor.

The housing may be any housing of an electrical device such as, for example, a cell phone, a smart phone, a smart watch, a portable computer, in particular, a tablet computer, or the like. The housing should have no openings other than the overflow opening, such as slots, crevices, or the like, through which a significant gas exchange between the inner space of the housing and the space outside of the housing might take place. Most electronic devices of the type of smart phones or cell phones already include such a sealed housing, since even today there are strict demands regarding dust-, water-, or moisture-tightness. Therefore, the refinement according to the present invention for a controlled gas exchange with the aid of a pumping motion may be implemented very easily and cost-effectively.

The gas flow flowing through the overflow opening past the sensor may be set to a desired quantity in a targeted manner by targeted deflection of the movable outer wall of the housing with the aid of an actuator. In this way, predetermined general conditions may be set on the sensor in a targeted manner. It is thus possible to operate the sensor under preferably optimal general conditions.

Interferences or even errors due to fluctuating and, in particular, unknown flow conditions upstream from the sensor may thus be avoided.

According to one specific embodiment, the movable outer wall of the housing includes a flexible wall. Such a flexible wall may be implemented, for example, by a flexible plastic element or the like. It is possible, in particular, that the movable, flexible outer wall of the housing is fixedly connected to the remaining housing at the transition to the remaining housing. In this way, a particularly hermetically sealed housing may be implemented.

According to one alternative specific embodiment, the movable outer wall of the housing includes a rigid wall. A sealing element may be situated between the movable outer wall of the housing and the rest of the housing. Due to this additional sealing element, a high degree of hermeticity may be ensured even in the event of a relative movement of the movable outer wall with respect to the rest of the housing.

According to another specific embodiment, the control device is designed to activate the sensor. In particular, the control device may activate the sensor when the actuator is energized. In this way, it is possible that the sensor actively carries out a detection when the pumping motion is carried out with the aid of the actuator due to the deflection of the movable outer wall. By synchronizing the actuator and the sensor, the detection by the sensor may be limited to those periods in which a pumping motion is actually carried out. In this way, on the one hand, it is possible to avoid erroneous detection in a period in which no pumping motion is carried out. On the other hand, energy is also saved by deactivating the sensor in those periods in which no pumping motion is carried out.

According to another specific embodiment, the actuator is designed to deflect the movable outer wall of the housing at a predetermined rate. The gas flow flowing through the overflow opening may be controlled via the targeted deflection of the outer wall. In this way, the gas flow through the overflow opening may be easily set to a predetermined gas flow, which is particularly suitable for detection with the aid of the sensor.

According to another specific embodiment, the overflow opening is designed to limit a volume flow through the overflow opening to a predetermined value. The gas flow may be set in a particularly easy manner by adjusting the volume flow of the gas flow flowing through the overflow opening.

According to another specific embodiment, the actuator is designed to deflect the movable outer wall from a rest position. The gas or particle measuring device also includes a restoring device. This restoring device is designed to restore the movable outer wall of the housing from a deflected position into the rest position. In this way it is possible that the actuator only actively deflects the movable outer wall of the housing, while the outer wall is restored into the rest position when the actuator is not actively energized.

For example, the restoring device may be a spring element or the like. Alternatively, it is also possible that the deflected movable outer wall is restored by prestressing the movable outer wall.

According to another specific embodiment, the overflow opening includes a first valve, which is designed to suppress a gas flow from the inner space to the space outside of the housing. Only one gas flow may thus flow through the overflow opening in one direction, namely, from the space outside to the inner space. In this case, the housing may include a further opening having a second valve, the second valve being designed to suppress a gas flow from the space outside to the inner space of the housing. Only one gas flow may thus flow through the further opening from the inner space to the space outside of the housing. It is thus possible that, if the sensor is situated in the proximity of the overflow opening, only one gas flow, in particular, the ambient air, is detected, which flows from the outside of the housing into the inner space.

According to another specific embodiment, the housing of the gas or particle measuring device also includes a chamber, which is connected to the overflow opening. The chamber may include at least one partially flexible outer wall. The gas exchange thus takes place during the pumping motion with the aid of the actuator between the space outside of the housing and the inner space of the chamber. Ambient air is thus prevented from penetrating the further inner space of the housing outside the chamber.

According to another specific embodiment, the gas or particle measuring device may include an additional sensor. This additional sensor may be a sensor for detecting a predetermined event or predetermined surroundings conditions. For example, the additional sensor may be a moisture sensor. However, acceleration sensors or the like are also possible. If these sensors detect a predetermined event such as, for example, the moisture exceeding a predetermined value, the deflection of the outer wall by the actuator may be suppressed. It may thus be ensured that in a hazardous situation, such as, for example, moisture in the space outside of the housing, no pumping motion takes place, which might possibly result in damage to the elements in the inner space of the housing.

According to another specific embodiment, the device includes a display unit, an operating unit, a transceiver unit, a control unit, an evaluation unit, and/or a power supply unit, which are situated in the housing of the device. In particular, the present invention may thus include an electronic device, such as, for example, an entertainment electronics device into which gas or particle measurement corresponding to the gas or particle measuring device is integrated.

The above embodiments and refinements may, if meaningful, be combined in any desired way. Further possible embodiments, refinements and implementations of the present invention include also combinations of features of the present invention not explicitly mentioned previously or below with respect to the exemplary embodiments. In particular, those skilled in the art will also add individual aspects as refinements or supplements to the particular basic form of the present invention.

### BRIEF DESCRIPTION OF THE DRAWINGS

The present invention is described in greater detail below based on the exemplary embodiments illustrated in the schematic figures.

FIG. 1 shows a schematic illustration of a cross section through a gas or particle measuring device according to one specific embodiment.

FIG. 2 shows a schematic illustration of a cross section through a gas or particle measuring device according to another specific embodiment.

FIG. 3 shows a schematic illustration of a cross section through a gas or particle measuring device according to yet another specific embodiment.

FIG. 4 shows a schematic illustration of a top view of a gas or particle measuring device according to one specific embodiment.

FIG. 5 shows a schematic illustration of a cross section through an electronic device including a gas or particle measuring device according to one specific embodiment.

FIG. 6 shows a schematic illustration of a flow chart on which a method for gas or particle measurement according to one specific embodiment is based.

### DETAILED DESCRIPTION OF EXAMPLE EMBODIMENTS

Unless otherwise stated, in all figures, identical elements or elements and devices having the same function are provided with the same reference numerals.

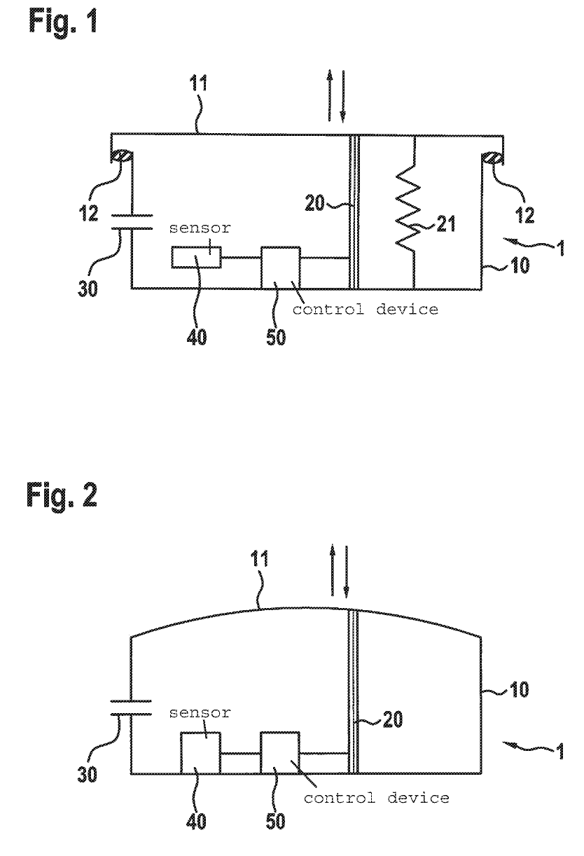

FIG. 1 shows a schematic illustration of a device 1 for gas or particle measurement according to one specific embodiment. Device 1 includes a housing 10. This housing 10 includes at least one outer wall 11, which is movable relative to the rest of the housing. For example, housing 10 may be the housing of a cell phone, a smart phone, a tablet computer, or of any other electronic device, in particular, of a portable electronic device. With the exception of movable outer wall 11, housing 10 is preferably formed by rigid, i.e., non-flexible or at least essentially non-flexible outer walls. Outer walls of this type may be formed, for example, by a metallic frame, plastic elements, and/or glass panels. Movable outer wall 11 is, in this exemplary embodiment, not fixedly connected to the remaining part of housing 10. Rather, a relative movement of both parts may occur at the connection points between movable outer wall 11 and the remaining parts of housing 10. A sealing element 12 is situated at the transition between movable outer wall 11 and the remaining part of housing 10. For example, this sealing element 12 may be a bellows, a rubber seal, or the like.

Housing 10 includes an overflow opening 30. This overflow opening 30 may be, in the simplest case, an opening having a predetermined size. Overflow opening 30 may have, for example, a circular, rectangular, or also any other shape. If necessary, a filter element (not shown here) may also be situated at overflow opening 30. Using such a filter element, it may be ensured that no undesirable substances, in particular, no particles beyond a predetermined size, may penetrate into the inner space of housing 10. With the exception of overflow opening 30, housing 10 has no, or at least no other significant, openings through which a gas exchange between the inner space of housing 10 and the space outside of housing 10 could be possible. The overflow opening may be situated, as FIG. 1 shows, at an edge of housing 10, for example. In addition, overflow opening 30 may also be situated in any other position of housing 10. For example, the overflow opening may be situated in movable outer wall 11 or on the side of the housing opposite to outer wall 11. Basically it is also conceivable that an already existing opening, such as, for example, an opening for a microphone or the like, be used as overflow opening.

Device 1 for gas or particle measurement also includes an actuator 20, which is designed to deflect movable outer wall 11 of housing 10. Actuator 20 is preferably situated in the inner space of housing 10. Actuator 20 may deflect movable outer wall 11 of housing 10 in the direction of the arrow. If movable outer wall 11 of housing 10 in FIG. 1 is deflected upward, the volume of the inner space of housing 10 increases. For equalizing the pressure between the inner space and the space outside of housing 10, a gas, in particular, the ambient air, flows from the outside into the inner space of housing 10. The gas flows through overflow opening 30 and further on past a sensor 40. Sensor 40 is able to detect a substance in the gas flowing through overflow opening 30 and past sensor 40. This may be, for example, the detection of one or multiple predetermined gas(es). For example, sensor 40 may be a sensor for nitrogen, oxygen, ozone, carbon dioxide and/or any other gas. In addition, sensors for detecting particles in a gas, in particular, particles of a predetermined size or of a predetermined size range are also possible. Sensors of this type for detection of particles are able to detect a quantity or a concentration of particles in a gas flowing past. Further sensors, for example, sensors for detecting moisture or the like, are also possible.

By adjusting the size or the geometry of overflow opening 30 and/or by setting the rate at which movable outer wall 11 is deflected by actuator 20, the gas flow flowing through overflow opening 30 past sensor 20 may be set.

Movable outer wall 11 of housing 10 may be deflected by actuator 20 to a maximum deflection. Movable outer wall 11 of housing 10 may subsequently be deflected in the opposite direction. In this way, the volume in the inner space of housing 10 is reduced and gas or air in the inner space of housing 10 flows through overflow opening 30 out into the space outside. Also in this case, it may be possible for sensor 40 to analyze the gas flowing past. Alternatively, it is also possible to carry out the analysis of the gas flowing past only when the gas flows from the space outside to the inner space and not to carry out any detection by sensor 40 when the flow direction is reversed.

For moving movable outer wall 11 in FIG. 1 downward, i.e., for a movement of movable outer wall 11 to reduce the volume in the inner space of housing 10, movable outer wall 11 may be moved actively by actuator 20. Alternatively, it is also possible to carry out this movement with the aid of an additional restoring element 21. In this case, movable outer wall 11 is deflected by actuator 20 from its rest position by actively energizing actuator 20, while deflected movable outer wall 11 is moved back into the rest position by restoring element 21 when actuator 20 is not actively energized. While movable outer wall 11 is restored by restoring element 21, energy may also be recovered if necessary with the aid of a generator element (not illustrated here). Actuator 20 may be any element for deflecting movable outer wall 11. In particular, piezoelectric elements or any other electromechanical elements are conceivable, for example.

If a larger quantity of gas is required for the analysis of a substance with the aid of sensor 40 than may be achieved by a one-time deflection of movable outer wall 11 by a single pumping motion with the aid of actuator 20, it is also possible that a predetermined number of consecutive deflections of movable outer wall 11 by actuator 20 takes place in order to achieve a predefined volume of a gas for the analysis by sensor 40.

Actuator 20 is energized, for example, by a control device 50. Control device 50 may synchronize the analysis by sensor 40 and the deflection of movable outer wall 11 by actuator 20, in particular. For example, sensor 40 may be activated whenever actuator 20 moves movable outer wall 11. For example, sensor 40 may be activated when the volume in the inner space of housing 10 is either increased or reduced by the movable outer wall 11 of housing 10. Alternatively, it is also possible to activate sensor 40 only when actuator 20 moves movable outer wall 11 of housing 10 outward, i.e., when the volume in the inner space of housing 10 is increased, i.e., a gas flow takes place from the space outside into the inner space of housing 10.

If necessary, device 1 for gas or particle measurement may also include further sensors (not illustrated), which detect further parameters, in particular, further surroundings parameters. For example, moisture or water may be detected in the outside space around housing 10 with the aid of a moisture sensor. When moisture or water is detected in the outside space around housing 10, a deflection of movable outer wall 11 of housing 10 may be subsequently suppressed if necessary. In this way, it may be ensured that, in the event of a high degree of moisture or water in the outside space, this moisture or water may not penetrate into the inner space of housing 10 through overflow opening 30, which may damage the other components in the inner space of housing 10. Additionally or alternatively, a possible vibration may also be detected by an acceleration sensor and then also a deflection of the movable outer wall may be suppressed if necessary. Furthermore, the position of device 1 for gas or particle measurement may also be detected with the aid of a position sensor. For example, the gas or particle measurement may be activated only when device 1 for gas or particle measurement is in one or multiple predetermined location(s). Alternatively, the measurement may also be activated whenever at least a predetermined distance is detected between a position of a previous measurement and the present position of device 1. In addition, the measurement at predetermined points in time or in predetermined time intervals may also be set with the aid of a timer.

FIG. 2 shows another specific embodiment of a device 1 for gas or particle measurement. This specific embodiment is mostly identical to the specific embodiment of FIG. 1. The specific embodiment according to FIG. 2 differs from the specific embodiment according to FIG. 1 only in that movable outer wall 11 is a flexible outer wall. Flexible, movable outer wall 11 may be connected to the remaining part of housing 10 fixedly and, in particular, hermetically. An additional sealing element may thus possibly be dispensed with. Deflection of movable outer wall 11 preferably takes place in the area around the center of movable outer wall 11. In this way, movable outer wall 11 may be deflected from a rest position.

Deflection preferably takes place by arching movable outer wall 11 outward with the aid of actuator 20.

However, movable outer wall 11 may also be arched inward with the aid of actuator 20. The volume of the inner space of housing 10 may also be increased or reduced in this way. Flexible, movable outer wall 11 may possibly be prestressed, which draws flexible movable outer wall 11 back into the rest position when movable outer wall 11 is not actively deflected by actuator 20.

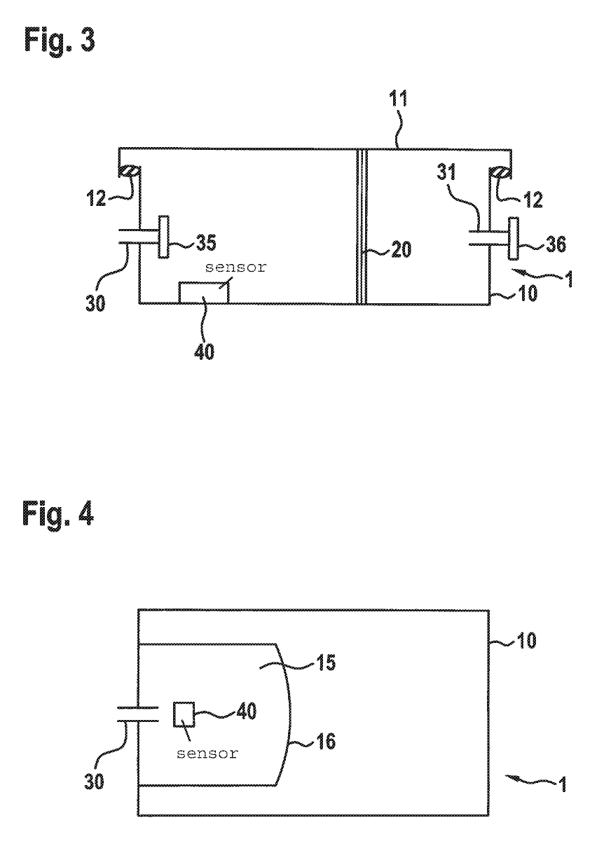

FIG. 3 shows another specific embodiment of a device 1 for gas or particle measurement. Although movable outer wall 11 is illustrated here as a rigid outer wall similar to the exemplary embodiment according to FIG. 1, this exemplary embodiment may also be implemented with a flexible, movable outer wall according to FIG. 2. The specific embodiment according to FIG. 3 differs from the previous specific embodiments in particular, in that housing 10 has at least one further opening 31 in addition to overflow opening 30. Both overflow opening 30 and further opening 31 may be designed in such a way that only one gas flow is possible in one direction. For example, a first valve 35 may be situated on overflow opening 30, and a second valve 36 may be situated on further opening 31. It may thus be achieved, for example, that only one gas flow from the space outside to the inner space of housing 10 takes place through overflow opening 30, while valve 35 prevents a gas from flowing from the inner space to the space outside of housing 10. Similarly, it is possible to enable only a gas flow from the inner space to the space outside of housing 10 and to prevent a gas exchange from the space outside to the inner space of housing 10 with the aid of second valve 36 on further opening 31. In this way, it may be ensured that always only one gas flow takes place at any time from the space outside to the inner space of housing 10 through overflow opening 30, and thereby gas flows past sensor 40 situated near overflow opening 30.

By adjusting the cross-section or the rate of movement of actuator 20, different volume flows may be set if necessary for the inflow of gas into the inner space of housing 10 and the outflow of gas from the inner space to the space outside of housing 10. In other words, it may be made possible that the gas flows at a higher or lower rate into the inner space than from the inner space to the space outside. If necessary, further opening 36 may correspond to an opening for a microphone of a cell phone or the like. In this case, for example, during the outflow of the gas from the inner space to the space outside, a microphone channel may be “blown out,” i.e., cleaned with the aid of the gas flowing past.

In addition, in all previously described specific embodiments, a pumping motion is possible, for example, via a very rapid, strong deflection of outer wall 11 by suitably energizing actuator 20, which results in a high volume flow through overflow opening 30 and/or further opening 31, and if necessary, removing possible dirt at these openings in the process.

FIG. 4 shows another specific embodiment for a gas or particle measuring device. This specific embodiment may be combined with basically any of the previously described specific embodiments. It differs from the previous specific embodiments only by the fact that an additional chamber 15 is situated in housing 10. This additional chamber 15 separates one area of the inner space of housing 10 from the rest of the inner space of housing 10. A gas may flow from the space outside through overflow opening 30 into chamber 15. Chamber 15 has at least one flexible wall 16. Thus, if movable outer wall 11 of housing 10 is deflected by actuator 20, initially a pressure differential is created between the inner space and the space outside of housing 10. This pressure differential thus results in a deflection of flexible wall 16 of chamber 15. A gas or the air may thereupon flow from the space outside into the inner space of chamber 15 through overflow opening 30. Sensor 40 may also detect a substance in chamber 15. Since chamber 15 separates the inner space of chamber 15 connected to the space outside via overflow opening 30 from the remaining inner space of housing 10, a possibly harmful substance, such as moisture, for example, or an aggressive gas, may not penetrate the rest of the inner space of housing 10 and thus result in possible damage to the components in housing 10.

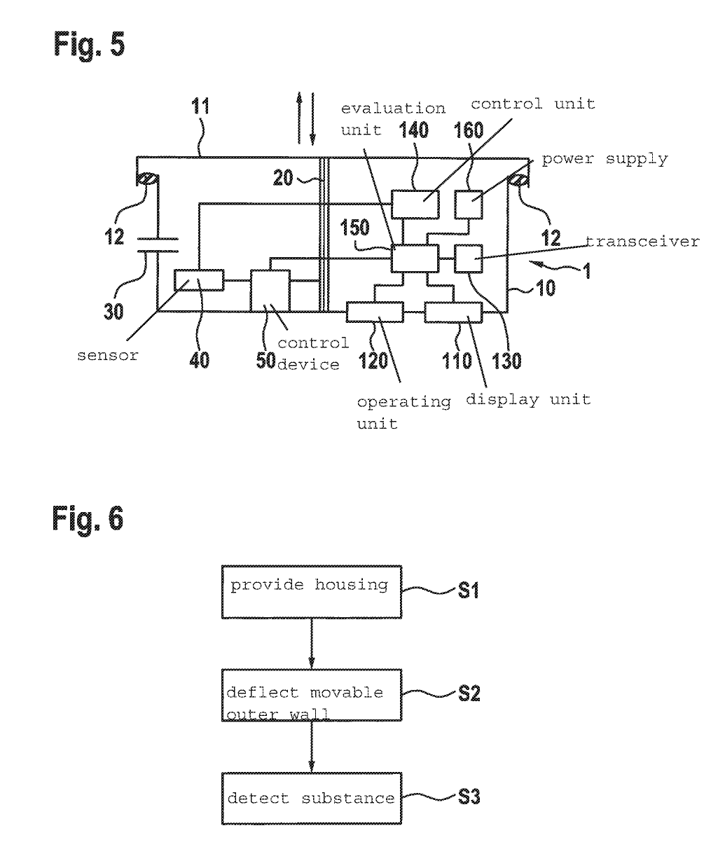

FIG. 5 shows a schematic illustration of an integration of a gas or particle measuring device into an electronic device. The electronic device may be, for example, a device of entertainment electronics. The integration of the gas or particle measuring device according to all previously described specific embodiments in electronic devices of this type is basically possible. In particular, integration into cell phones, smart phones, smart watches, tablet computers, and any other portable devices of entertainment electronics is possible. Overflow opening 30 is preferably not situated in movable outer wall 11 of housing 10.

The electronic device includes, in addition to the components named in connection with the above-named exemplary embodiments, a display unit 110, an operating unit 120, a transceiver unit 130, a control unit 140, an evaluation unit 150, and a power supply unit 160. Each of the above-mentioned units may also include suitable electronics, in particular, microprocessor-controlled electronics, which are designed to control the corresponding unit. Even when, for better understanding of the present invention, the additional units of an electronic device are not expressly mentioned in the above exemplary embodiments of FIGS. 1 through 4, it is still possible that each of these specific embodiments includes further units 110 through 160.

Display unit 110 may be any display, such as, for example, an OLED or TFT display. In addition to displays of conventional functions, display unit 110 may also be designed to display measuring results of the gas or particle measuring device. Operating unit 120 may be any type of suitable input device. For example, user input may take place with the aid of a keyboard or a touch screen. In addition, further input methods such as, for example, voice input using a microphone, are also possible. By using a touch screen or the like, display unit 110 and operating unit 120 may be also combined into one unit.

Transceiver unit 130 may be a communication unit, which is designed to exchange data with an external partner. For example, transceiver unit 130 may be a wireless interface. In particular, transceiver unit 130 may carry out wireless communication with the aid of GSM, UMTS, LTE, WLAN, Bluetooth, ZigBee, infrared, or the like. Transceiver unit 130 may be designed, in particular, to transmit measuring results of the gas or particle measuring device and/or to receive commands for a gas or particle measurement by the gas or particle measuring device. Control unit 140 may be designed to control the functions of the electronic device and in particular, also the functions of the gas or particle measuring device. For example, control unit 140 may include a microprocessor-controlled electronic circuit for this purpose. Evaluation unit 150 may include an electronic circuit, in particular, a microprocessor-controlled electronic circuit. Evaluation unit 150 may be designed to receive data from different components of the electronic device and to evaluate or analyze them. In particular, evaluation unit 150 may receive and process data from different sensors, such as, for example, sensor 40 of the gas or particle measuring device.

Power supply unit 160 may be, for example, a battery or a rechargeable battery. In addition, other components such as, for example, photovoltaic modules or the like are also possible, which are designed to provide electrical power. Furthermore, a cable-bound power supply or power transmission with the aid of electric, magnetic, or electromagnetic fields, is also possible. The individual components of the electronic device and, in particular, the components of the gas or particle measuring device, may thus also be supplied with electrical power.

FIG. 6 shows a schematic illustration of a flow chart serving as a basis for a gas or particle measuring method according to one specific embodiment. In step S1, a housing 10 is initially provided. This housing 10 includes an outer wall 11, which is movable relative to the remaining housing 10. Furthermore, the housing includes an overflow opening, which is designed to enable a gas exchange between the inner space and the space outside of housing 10. Furthermore, an actuator 20 is provided, which is designed to deflect outer wall 11 of housing 10. In step S2, movable outer wall 11 of housing 10 is deflected by actuator 20, and thereupon in step S3, a substance is detected in the gas flow flowing through the overflow opening. The method described herein may be used, in particular, in an electronic device, in particular a device of entertainment electronics, as it was described, for example, for example, in connection with FIG. 5.

In summary, the present invention relates to a device and a method for gas or particle measurement in a portable electronic device. The portable device includes a housing, which has an almost gas- and air-tight design, except for an overflow opening. In particular, a display unit, an operating unit, a transceiver unit, and optionally a power supply unit may be integrated into the housing. Furthermore, the housing may also include the necessary electronics such as, for example, a control unit, an evaluation unit, etc. By deflecting a movable outer wall of the housing with the aid of an actuator, the volume of the inner space of the housing may be increased or reduced. A gas exchange may then take place between the inner space and the space outside of the housing through the overflow opening in the housing, and a possible substance in a gas flow, in particular, a predetermined gaseous component or a particle concentration, may be analyzed during this gas exchange.

What is claimed is:

1. A device for gas or particle measurement, comprising:
a housing including a movable outer wall, which is movable relative to the remaining outer walls of the housing, and including an overflow opening, which is designed to enable a gas exchange between an inner space and a space outside of the housing;
an actuator designed to deflect the movable outer wall of the housing;
a control device designed to energize the actuator; and
a sensor designed to detect a substance in a gas flow flowing through the overflow opening.

2. The device as recited in claim 1, wherein the movable outer wall of the housing includes a flexible wall.

3. The device as recited in claim 1, wherein the movable outer wall of the housing includes a rigid wall, and a sealing element is situated between the movable outer wall of the housing and the remaining housing.

4. The device as recited in claim 1, wherein the control device is designed to activate the sensor when the actuator is energized.

5. The device as recited in claim 1, wherein the actuator is designed to deflect the movable outer wall of the housing at a predetermined rate.

6. The device as recited in claim 1, wherein the overflow opening is designed to limit a volume flow through the overflow opening to a predetermined value.

7. The device as recited in claim 1, wherein the actuator is designed to deflect the movable outer wall of the housing from a rest position, and the device also includes a restoring device, which is designed to restore the movable outer wall of the housing into the rest position.

8. The device as recited in claim 1, wherein the overflow opening includes a first valve, which is designed to suppress a gas flow from the inner space to the space outside of the housing, and the housing includes a further opening including a second valve, which is designed to suppress a gas flow from the space outside to the inner space of the housing.

9. The device as recited in claim 1, wherein the housing also includes a chamber, which is connected to the overflow opening, and the chamber includes an at least partially flexible wall.

10. The device as recited in claim 1, further comprising at least one of the following, situated in the housing:
a display unit;
an operating unit;
transceiver unit;
a control unit;
an evaluation unit; and
a power supply unit.

11. A method for gas or particle measurement, comprising:
providing a housing including an outer wall which is movable with respect to the rest of the housing and including an overflow opening, which is designed to enable a gas exchange between an inner space and a space outside of the housing;
providing an actuator designed to deflect the movable outer wall of the housing;
deflecting the movable outer wall of the housing by energizing the actuator; and
detecting a substance in a gas flow flowing through the overflow opening.
